(19) United States
(12) Patent Application Publication
(10) Pub. No.: US 2011/0251127 A1
(43) Pub. Date: Oct. 13, 2011

(54) Inactivation of infectious agents in plasma proteins by extreme pressure

(76) Inventor: Richard C.K. Yen, Yorba Linda, CA (US)

(21) Appl. No.: 13/134,043

(22) Filed: May 26, 2011

Related U.S. Application Data
(62) Division of application No. 11/804,428, filed on May 18, 2007, now abandoned.
(60) Provisional application No. 60/802,296, filed on May 22, 2006.

(51) Int. Cl. A61K 38/36; A61P 7/02; C07K 14/75

(52) U.S. Cl. 514/13.7; 428/402

(57) ABSTRACT

A method of inactivation of infectious agents in a fluid containing plasma protein and potentially containing at least one infection agent which includes unique steps of placing the fluid in a container which is resistant to leakage under high pressure, placing the container in a compression chamber, pressurizing the fluid inside the container to a pressure sufficient to inactivate the potential infectious agent, pressurizing the fluid under a high pressure for a time duration sufficient to inactivate the potential infectious agent and pressurizing the fluid under the high pressure at an initial temperature that does not inactivate coagulation factors under the conditions. The present invention also includes a fluid containing plasma proteins which is pressurized to inactivate infectious agents, the plasma proteins containing serum albumen and at least one coagulation factor.

### The present invention is a divisional of co-pending patent application Ser. No. 11/804,428 filed on May 18, 2007.

### BACKGROUND OF THE INVENTION

1. Field of the Invention

The present invention relates to the field of plasma and blood products derived from plasma which are used for emergency resuscitation and chronic administration to patients. It is absolutely essential that they are free of infectious agents.

2. Description of the Prior Art

Various methods had been employed to inactivate potential infectious agents present in plasma or plasma-derived products. Heat, for example, had been used to treat human serum albumin with success (Edsall J. T. “Stabilization of Serum Albumin to Heat, and Inactivation of the Hepatitis Virus” Vax Sang 67:1-86, 1984.) Recently, Alpha Therapeutic Corporation announced having obtained clearance from the U.S. Food and Drug Administration to include a heat treatment step for its solvent detergent treated Antihemophilic Factor (AHF) product (Mar. 19, 1997, PR Newswire.)

One common method of viral inactivation, particularly effective against lipid-enveloped viruses such as HIV, hepatitis B (HBV) and hepatitis C viruses (HCV) is the “solvent/detergent” (S/D) method. Use of this method to treat hemophilia concentrates since 1985 had resulted in over 75,000 man-years of treatment without a single report of HBV, HCV or HIV transmission (Kiss J. “Viral Inactivation of Plasma and Plasma Products” Transfusion Medicine Update, the Institute For Transfusion Medicine, July 1994.) However, the S/D method may not work well with non-enveloped viruses such as hepatitis A.

Other methods had been disclosed using various chemicals, e.g. riboflavin (Goodrich R. P. “The Use of Riboflavin for the Inactivation of Pathogens in Blood Products” Vox Sang 78 Suppl 2:211-5, 2000) or caprylate (Johnson A. et al., “Low pH, Caprylate Incubation as a Second Viral Inactivation Step in the Manufacture of Albumin. Parametric and Validation Studies” Biologicals 31(3):213-21, 2003.) Others tried phenothiazines such as methylene blue (Specht K. G. “The Role of DNA Damage in PM2 Viral Inactivation by Methylene Blue Photosensitization” Photochem Photobiol., 59(5):506-14, 1994) to some effect. Thio compound had also been used (Hofschneider, Peter, US Patent Application 20020040057 “Use of Thiol Compound in Viral Inactivation” Apr. 4, 2002).

Sometimes multiple regiments were used, e.g. in the Press Release (Jul. 21, 2003, Research Triangle Park, N.C.) Bayer Biological Products announced a method of effectively inactivating Monkeypox virus (an “envelop” virus) by using a combination of solvent/detergent, heat treatment (followed by extraction using acetone), incubation in acidic solutions, and then inactivation with caprylate.

Still others tried gamma irradiation. House et al., reported in the Canadian Journal of Microbiology 36, 737-740, 1990, that 25 kilogray of gamma irradiation could inactivate 6 logs of Bovine Viral Diarrhea Virus. Hanson, Grand Foster L. (“Viral Safety in Cell Culture Use” Art to Science, 16: 1-7, 1997) and Daley J. P et al (“Virus Inactivation by Gamma Irradiation of Fetal Bovine Serum” Focus 20: 86-88, 1998) also used gamma radiation as a means of viral inactivation.

Aphios Corp (216 Sylvia Street, Arlington, Mass. 02174) disclosed a method using carbon dioxide raised above its critical temperature and pressure (using low pressure and short processing time, not high pressure) called “critical fluid Inactivation.”

US Patent Application No. 20050051497 disclosed a method of using ozone for sterilization (Joseph S. Latino and Steven A. Keyser, “Viral Inactivation Using Ozone” Mar. 10, 2005).

Inherent in all of the above methods are problems associated with (a) destruction of the biological activity of the desired medical or commercial product along with inactivation of the targeted infectious agents, (b) the potential of creating neo-antigenicity in the treated biological product by the method of treatment, (c) the need to remove residuals of the treatment chemical, (d) the need to show the method of removal of the residues of the treatment chemical is itself safe and complete, (e) costs in time, labor, machinery, packaging, scale-up involved.

Addition of stabilizers occasionally preserved the biological activity of sensitive material, such as coagulation factor VII, after pasteurization for 10 hours at 60 degree C. (Rezvan H., et al. “Inactivation of Poliovirus Type 1 and HSV-1 in Human Coagulation Factor VII Concentrate by Pasteurization” http://www.ams.ac.ir/AIM/0141/rezvan0141.html.)

However, what single stabilizer to use, in what concentration, or in combination with what other stabilizers, remains more of an art than science. For example, sucrose added at 70% to a solution of factor VII concentrate led to only a 28% recovery of activity after pasteurization (the appearance of the treated material being described as “opalescent’.) Addition of glycine to 20% concentration in a solution containing factor VII resulted in 23% recovery of activity (the appearance of the treated material was “opalescent.”) However, a mixture of 70% sucrose plus 20% glycine resulted in recovery of 80% of the factor VII activity (the appearance of the material after pasteurization was “Clear”, table 1.) Addition of sodium citrate (commonly used for anticoagulation of the collected plasma) from 0.5 M to 2.0 M did not lend any protection to the factor VII during pasteurization; the treated material became a gel which rendered it totally useless for infusion into patients.

Moreover, addition of stabilizers had the adverse effect of protecting the virus to be inactivated. For example, heat treatment of poliovirus type 1 for 10 hours at 60 degree C. in an isotonic buffer solution resulted in complete inactivation within 1 hour, while inactivation was not complete until the 8^{th} hour when the virus was place in a “stabilized” factor VII solution. (FIG. b 2.)

High (hydrostatic) Pressure Processing (HPP) has been used in the food industry (Hoover, D. G. “Nonthermal Processing Division Lecture: Viral Inactivation by High Hydrostatic Pressure” Session 88, Inactivating Pathogens, Parasites, and Viruses Using High Pressure and Other Emerging Nonthermal Technologies, 2004 IFT Annual Meeting, Jul. 12-16, Las Vegas, Nev.) The material (such as raw oyster or other sea food) to be treated is typically placed inside a plastic bag, sealed, which itself is then immersed in a “compressor” fluid (such as water) inside a compression or pressure chamber. After the compression chamber is closed, hydraulic pressure (such as up to 600 or more MPa) is applied to the water, which under correct conditions can lead to sterilization of the material inside the plastic bag. The advantage is that no chemical needs to be added to the product to be treated and therefore no need for its removal. Another advantage is that pressure applied within the pressure chamber is spread uniformly to all surfaces and the sealed material immersed in the compressor fluid, allowing terminal sterilization of biological material packaged in pliable bottles or containers (such as plastic bags or vials.)

Examples of using HPP included inactivation of noroviruses and hepatitis A (a non-enveloped virus) in raw oysters (Grove S. F. et al., “Inactivation of Foodborne Viruses of Significance by High Pressure and Other Processes” J. Food Prot. 69(4):956-68, 2006.) The authors pointed out that “Viruses have demonstrated a wide range of sensitivities in response to high hydrostatic pressure. Viral inactivation by pressure has not always been predictable based on nomenclature and morphology of the virus.” Therefore just because certain viruses had been inactivated successfully in one medium does not guarantee its success in another biological or non-biological fluid.

Selected picornaviruses had already been inactivated by HHP (Kingsley D. H, et al., “Inactivation of Selected Picornaviruses by High Hydrostatic Pressure” Virus Res. 102(2):221-4, 2004.) Viruses such as Aichi virus, human parechovirus-1 (HPeV-1) and the coxsackievirus strains A9 and B5 were inactivated in minimum essential growth medium supplemented with 10% fetal bovine sera. For HPeV-1, a 5-min treatment at 600 MPa achieved a 4.6 log reduction in Tissue Culture Infectious Dose-50%, TCID(50); but the other two viruses remained fully infectiously after a similar treatment. The data showed that different viruses (even all classified as picornavirus) have widely variable pressure inactivation thresholds. The effect of such high hydrostatic pressure on the sera is not known. Plasma (containing coagulation factors) had not been tested.

Chen H et al. disclosed a method of inactivation of feline calicivirus using a combination of different temperature and treatment times (Chen H. et al., “Temperature and Treatment Time Influence High Hydrostatic Pressure Inactivation of Feline Calicivirus, a Norovirus Surrogate” J. Food Prot. 68(11):2389-94, 2005.) Feline calicivirus (FCV) is a propagable virus that is genetically related to the nonpropagable human noroviruses and therefore suitable for evaluation of HPP inactivation. The temperature selected were −10, 20 (room temperature) and 50 degree C., with treatment time from 2 to 4 minutes. The data showed that FCV could be inactivated by the lowest and the highest temperatures, was most resistant to pressure inactivation at 20 degree C. Therefore the intuitive assumption that “higher” temperature (20 degree compared to −10 degree C.) should lead to a greater extent of viral inactivation (than when HPP was applied at 31 10 degree C.) is not always correct. In fact, inactivation by a 4 minute treatment at 200 MPa at −10 degree C. led to a 5.0 log reduction in viral titer compared to only a 4.0 log reduction when treatment was performed at 50 degree C. The data showed that effective treatment methods (whether as a combination of temperature and treatment time, or as a combination of additional factors, such as inclusion of chemicals or other physical means) is not predictable, nor obvious. The study also focused only on the condition necessary to achieve inactivation of the virus and did not study its effect on the biological media, which may be affected in an unpredictable manner, particularly in biologically sensitive molecules including coagulation molecules, which may not function when “bent out of shape.”

Multiple factors affected the effectiveness in viral inactivation using conventional methods, including (a) viral property (DNA/RNA, single/double strandedness, naked or enveloped,) (b) viral titer, (c) medium containing the virus, (d) pH, (e) salt concentration. (See Sullivan et al, “Inactivation of Thirty Viruses by Gamma Radiation” Applied Microbiology 22:61-65, 1971). Therefore, the conditions necessary for this new method of using HPP to successfully inactivate different viruses, bacteria, fungi, parasites or abnormal pathological proteins (e.g. prions), are not obvious.

Non-viral agents such as bacteria, fungus, parasites and the spores (and enzymes) from these organisms had been shown to be destroyed by HPP (Jordan C. N. et al. “Effects of High-Pressure Processing on in vitro Infectivity of Encephalitozoon cuniculi” J. Parasitol 91(6):1487-8, 2005.)

U.S. Pat. No. 6,537,601 (Voisin, “Process of Elimination of Bacteria in Shellfish and of Shucking Shellfish, March 25, 2003) disclosed a method to eliminate pathogenic bacteria using pressure up to 60,000 p.s.i (equals to 414 MPa) conducted at elevated temperatures in the range of 50 to 130 degree F. The patent did point out that the muscle proteins of oysters (including actin and myosin and connective tissues) do go through a “gelation transition” as a result of the disruption of non-covalent interactions in its tertiary protein structures under high pressure (page 12 of 15, under “Detailed Description of the Preferred Embodiment.”) Therefore it is expected that substantial loss of biological activity may occur in other biological molecules that are sensitive to changes in molecular shape. Voisin did not teach about preservation of biological activity of conformation-sensitive biological material such as coagulation factors.

### SUMMARY OF THE INVENTION

Plasma, or any of its derivatives in liquid form (or any conformation-sensitive biological material) is placed inside a plastic or pliable container which then is sealed with as little air trapped inside as possible. The container is placed inside a compressor fluid which is inside a compression chamber. After the compression chamber is closed, pressure is applied by a hydraulic pump or compressor until the desired pressure is achieved inside the compression chamber. After the desirable time (“holding time”) is achieved, pressure is released at a desirable rate from the compression chamber. Any infectious agent potentially present inside the plastic container would have been inactivated by the process. The compression chamber further provides a means to control the temperature of the compressor fluid for the protection of the biological material.

The method further anticipates addition to the plasma (or material to be treated) prior to pressurization, other chemicals (such as solvent/detergent) or material suitable for the protection of the biological activity of the plasma or for the enhancement of inactivation of infectious agents.

It has been found that three pulses of high pressure (up to 600 MPa) each holding for 5 minutes at an initial temperature of 5 degree C. had been effective in the inactivation of non-enveloped and enveloped viruses seeded in plasma, with more than 4 logs of inactivation, while preserving over 80% of the activity of coagulation factors, as compared to un-pressured control samples.

It has been further found that addition of solvent/detergent to the plasma before proceeding with a similar regiment of high pressure treatment can lead to a similar preservation of the activity of coagulation factors but with even greater effectiveness in the inactivation of infectious agents.

It has been further discovered that plasma proteins, whether as a single entity or as a combination of several coagulation factors can attach to protein spheres and that the resultant spheres can be treated by high pressure up to 600 MPa without loss of effectiveness in the treatment of bleeding in thrombocytopenic animals.

It has been further discovered that protein spheres without exposure to coagulation factors during the in vitro manufacturing process can be treated with high pressure to inactivate any potential infectious agents present in the protein solution used to manufacture the protein spheres or introduced during the filling and bottling process.

It has been further discovered that a novel method of treatment of hemophilia patient is effective by the infusion or injection of coagulation factors, whether as single purified molecules or as a combination of factors as in plasma, which had been treated by high pressure as disclosed in this Invention.

It has been further discovered that a novel method of treatment of thrombocytopenic patient is effective by the intravenous infusion of protein spheres coated with coagulation factor, whether as a single entity of purified molecules or as a combination of factors, after the spheres had been treated by high pressure as disclosed in this Invention.

Further novel features and other objects of the present invention will become apparent from the following detailed description, discussion and the appended claims.

### DETAILED DESCRIPTION OF THE PREFERRED EMBODIMENTS

Although specific embodiments of the present invention will now be described, it should be understood that such embodiments are by way of example only and merely illustrative of but a small number of the many possible specific embodiments which can represent applications of the principles of the present invention. Various changes and modifications obvious to one skilled in the art to which the present invention pertains are deemed to be within the spirit, scope and contemplation of the present invention as further defined in the appended claims.

### Experiment One

### Tolerance of Coagulation Factors Under Extreme High Hydrostatic Pressure at Initial Temperature of 5 Deg C.

Purpose: to evaluate the activity of a representative of coagulation factor (Factor I; fibrinogen) after repeated exposure to high pressure.

Material and Method: Fibrinogen was purchased from Sigma-Aldrich (derived from human plasma, negative for HIV and Hepatitis B surface antigen, containing about 15% sodium citrate and about 25% sodium chloride by weight.) A fibrinogen solution was prepared by dissolving 4 mg of powdered fibrinogen (with excipient) per ml of normal saline. Fresh plasma was donated by a healthy donor not under any medication and anti-coagulated with citrate. Fibrinogen activity was measured in a fibrometer (FibroSystem™)

Four plastic bags were filled with 2 ml each of the fibrinogen solution and sealed. Bag A served as control (kept in room temperature without pressurization.) Bag B, C, D were subjected to 600 MPa (megaPascal) at 5 degree C. (initially) with hold time of 5 minutes, once, twice and three times, respectively.

Four other plastic bags were filled with 2 ml of each of the fresh plasma from the donor. Bag E served as control (kept in room temperature without pressurization.) Bag F, G, H were subjected to 600 MPa (megaPascal) at 5 degree C. (initially) with hold time of 5 minutes, once, twice and three times, respectively.

Results: The concentration of active fibrinogen in Bag A was found to be 2.2 mg/ml. After pressurization the concentration of fibrinogen in Bag B, C, D were found to be 2.1, 2.0, 2.1 mg/ml, respectively. The concentration of fibrinogen in Bag E, F, G, H were 1.8, 1.8, 1.7, 1.8 mg/ml, respectively.

Comments: The data showed that up to three pulses of high pressure at 600 MPa did not substantially diminish the activity of a fibrinogen solution in normal saline, or in plasma medium. It is believed that if assayed for Factor VII, Factor VIII, vonWillibrand factor or any other coagulation factors, the data will support a similar conclusion with respect to the tolerance of these coagulation factors under similar conditions of high pressure treatment.

### Experiment Two

### Tolerance of Coagulation Factors Under Extreme High Hydrostatic Pressure at Initial Temperature of 20 and 30 Deg C.

Purpose: To evaluate the activity of coagulation factors after being subjected to 600 MPa at initial temperatures of 20 and 30 degrees C.

Material and Method: similar to that of Experiment One.

Results: The results were comparable to that of Experiment One.

Conclusion: Coagulation factors did not appear to have diminished activity after 3 pulses of high pressure up to 600 MPa, even with initial temperatures of 20 and 30 degrees C.

### Experiment Three

### Inactivation of Viruses Seeded in Plasma

Purpose: To evaluate if 3 pulses of high pressure will decrease the infectivity of a number of viruses by more than 4 logs.

Material and Methods: Six strains of viruses were seeded at a concentration of at least 20,000 infectivity titers/ml in separate bags containing fresh human plasma. The viruses were: (a) Pseudorabies Virus: PRV (b) Encephalomyocarditis Virus: EMCV ; (c) Human Immunodeficiency Virus, HIV; (d) Sindbis; (e) polio; (f) Vesicular Stomatitis Virus, VSV (which is an envelop virus.)

The bags were subjected to 3 pulses of pressure up to 600 MPa (hold time of 5 minutes) with initial temperature at 5 degree C. Fibrinogen concentrations in bags after pressurization and in a bag (control) not seeded with virus and not subjected to pressurization were measured as described in Experiment One.

Methods used for the detection of residual virus titer after pressurization were essentially equivalent to published methods: (a) for PRV: treated plasma is inoculated in PK-15 line, using the “cytopathic effect” CPE method, Tahir R A and Goyal S M, “Rapid Detection of Pseudorabies Virus by the Shell Vial Technique” J. Vet Diagn Invest, 7(2):173-6, 1995; (b) for EMCV: treated plasma is inoculated into BHK-21 cells, using the CPE method of detection, Kassimi et al., “Nucleotide Sequence and Construction of an Infectious cDNA Clone of an EMCV Strain Isolated from Aborted Swine Fetus” Virus Res. 83(1-2):71-87, 2002; (c) HIV: plaque assay done in MT4 cells, Iglesias-Sanchez and Lopez-Galindez C, “Each Genomic RNA in HIV-1 Heterozygous Virus Generate New Virions” Virology, 333(2):316-23, 2005; (d) Sindbis, cultured in BHK-21 cells using the PE method, expressed as log PEU/ml; Smit J. M. et al., “PE2 Cleavage Mutants of Sindbis Virus: Correlation Between Viral Infectivity and pH-dependent Membrane Fusion Activation of the Spike Heterodimer” J. Virol. 75(22):11196-204, 2001; (e) Polio, in vero cells, Haastrup E. et al. “Safety and Immunogenicity of a Booster Dose of Inactivated Poliovirus Vaccine Produced in Vero-cells” Vaccine, 22(8):958-62, 2004; VSV cultured in 293T cells, Saha M. N., et al. “Formation of Vesicular Stomatitis Virus Pseudotypes Bearing Surface Proteins of Hepatitis B Virus” J. Virol 79(19):12566-74, 2005.

Results: Assay of the bags containing different viruses separately showed that infectivity in all cases was completely eliminated. Fibrinogen activity remained at more than 90% of the control value.

Conclusion and Comments: Extreme hydraulic pressure up to 600 MPa (initial temperature at 5 degree C., 3 pulses) can inactivate non-enveloped as well as enveloped viruses in plasma for at least 4-logs of TCID50. Although in this experiment each kind of virus was inactivated in isolation (in separate bags) from each other, it is believed a mixture of different viruses in the same bag subjected to the same inactivation regiment will result in similar degrees of viral inactivation.

### Experiment Four

### Inactivation of Viruses in the Presence of Solvent/Detergent Added to Plasma

Purpose: To evaluate the effectiveness of viral inactivation under conditions that promote greater viral inactivation than Experiment 3, while preserving the activity of coagulation factors

Material and Method: The regiment of Experiment 3 was followed except for the following: (a) Solvent/Detergent (3% tri(n-butyl)phosphate (TNBP) and 1% Tween-80) was added to the plasma in each bag before the respective viruses were spiked into the plasma; (b) the concentration of virus in the spiked plasma was at least 40,000 infectious units per ml; (c) the initial temperature of the compressor fluid was 20 degree C.

Results: Assay of the bags containing different viruses separately showed that infectivity in all cases was completely eliminated. Fibrinogen activity remained at more than 80% of the control value.

Conclusion and Comments: Extreme hydraulic pressure up to 600 MPa (initial temperature at 20 degree C., 3 pulses) can inactivate non-enveloped as well as enveloped viruses in plasma for at least 4-logs of TCID50. Although in this experiment each kind of virus was inactivated in isolation (in separate bags) from each other, it is believed a mixture of different viruses in the same bag subjected to the same inactivation regiment will result in similar degrees of viral inactivation.

### Inactivation of Infectious Agents Among Coagulation Factors Associated with Solid or Semi-Solid Surfaces

While one approach of inactivation of infectious agents was to treat the solution containing coagulation factors, such as whole plasma or partially purified or pure coagulation factor solutions by extreme high hydrostatic pressure, other approaches are contemplated here.

One application of coagulation factors is the coating of protein particles or spheres with a single entity or a combination of coagulation factors so that the coated spheres can serve as artificial platelets or platelet substitutes. Prior art (U.S. Pat. No. 6,264,988 B1 “Fibrinogen-coated Microspheres”) described in detail how such spheres can be manufactured. It is contemplated in this Invention that the suspension containing spheres, residual protein molecules in solution, spheres coated with coagulation factors and residual coagulation factors still in solution can all be decontaminated from infectious agents by treatment of the suspension under high hydrostatic pressure.

Prior art describing how spheres are made all involved the addition of a detergent or surfactant to the protein solution before a desolvation agent (such as 70% ethanol) was added to create spheres from soluble albumin molecules. Those sphere suspensions prepared by the prior art had not been subjected to pressure nor evaluated after pressure treatment in thrombocytopenic rabbits. It is expected from the data presented in this Invention that such spheres after being coated with a fibrinogen solution in vitro, or coated with a combination of coagulation factors in vitro can be decontaminated by the extreme high pressure disclosed in this patent application.

Novel methods of making spheres without the addition of a detergent or surfactant to the protein solution are the subject of another patent disclosure and are described in a summary form below. All these spheres made with the novel manufacturing processes can be coated with a solution containing one entity of coagulation factor or a combination of coagulation factors, such as with whole plasma. These coated spheres can also be subjected to extreme high pressure and not affected in their medical efficacy. One medical efficacy is the shortening of bleeding time in thrombocytopenic rabbits, or a decrease in the volume of blood loss.

All of the above suspensions containing spheres made either with the prior art or with the novel disclosure here, after treatment by high pressure in the container, can then be infused into a patient for the treatment of thrombocytopenia or for prophylactic use to decrease the amount of anticipated blood loss or for treatment of active bleeding.

This patent application covers the use in patients of coagulation factors in solution which had been treated with high pressure for the purpose of inactivating any infectious agents present in the solution. Infectious agents will include any known or unknown bacteria and virus and other disease causing entities. Applications will include intravenous infusion or intramuscular injection to hemophilic patients lacking in any number of coagulation factors.

This patent application further covers the use in patients of suspensions of spheres or particles which had been subjected to high pressure for the purpose of inactivation of infectious agents introduced in any of the components and in any of the steps of manufacturing up to the step of pressure treatment.

In particular, this patent application covers the use in patients of suspensions of spheres coated with one coagulation factor or a number of different coagulation factors all of which had been subjected to high pressure for the purpose of inactivation of infectious agents present in the suspension. One example of such medical use will be the treatment of bleeding episode in thrombocytopenic patients of any number of etiologies, such as dilutional thrombocytopenia, idiopathic thrombocytopenia purpura, aplastic anemia, leukemia or lymphoma cancer patient or patients under chemotherapeutic treatment. Another example will be the prophylactic use of such a suspension in patients expected to have massive blood loss, such as in surgery or in countries where safe blood products are in extreme short supply, such as after a natural disaster, during war, or where blood borne infectious agents are so prevalent that there are not enough uninfected blood donors in the population. A third example of application for such pressure-treated spheres will be in patients with active bleeding from any cause.

### Brief Summary of the Various Novel Methods of Making Spheres without the Addition of Detergents in the Protein Solution

All of the following methods do not have added surfactant or detergent in the protein solution before the addition of a desolvation agent to form spheres. Some of the names may be similar to those described in the Prior Art (e.g. U.S. Pat. 6,264,988 B1) but the products made with these novel methods are different from those produced by the Prior Art. The name can refer to the method of production or the product produced by the method.

1. “Pre-link” means the crosslinking agent is added to the protein solution before the desolvation agent is added. In this method the crosslinking agent binds on sites on the protein molecules which are surrounded by water molecules with the protein molecule folded in the most natural state. Then at the addition of the desolvation agent, the protein molecules with attached crosslinking agents come together to form spheres.

2. “Mid-link” means the crosslinking agent is first premixed with the desolvation agent; and then the mixture is added to the protein solution. In this case the time of interaction of protein molecules with the crosslinking agent is of the same duration as that with the desolvation agent. In this method, the crosslinking molecules attach to some individual protein molecules in solution (surrounded by water of hydration) as well as those that have other protein molecules as their near-neighbors (partially surrounded by water of hydration.) Since the spheres are in the process of formation under the simultaneous action of the desolvation agent and the premixed crosslinking agent, it is conceivable that some protein molecules already have crosslinking agents attached (completely or partially) as they come together while other protein molecules had not yet reacted with any crosslinking molecules. It is also conceivable that additional crosslinking molecules will bind to the sphere after it is essentially formed. Since having another protein molecules as a near-neighbor (instead of water of hydration) may change the conformation of a protein molecule, the site of binding for the crosslinking agent may be different from those protein sites bound by the crosslinking agent under the “Pre-link” method mentioned above, or other methods to be mentioned below.

3. “Post-link” means the crosslinking agent is added after the desolvation agent had been added to the protein solution to form spheres. In this case the crosslinking agent is added to a turbid suspension of spheres which otherwise can redissolve if the desolvation agent is diluted or removed. In this case, the crosslinking molecule will bind onto sites on the protein molecules which have already been assembled as a sphere. The site of binding by the crosslinking agent may be different from those protein sites available for binding by the crosslinking agent when used under the “Pre-link” or “Mid-link” method mentioned above, or under other methods to be mentioned below.

4. “Bi-link” means the crosslinking agent is added in two separate steps. The first step involves adding a low concentration of crosslinking agent to the protein solution for a short time. This sub-stabilizing concentration of crosslinking agent is not sufficient to prevent resolubilization of the spheres upon dilution or removal of the desolvation agent but has the beneficial effect of preventing formation of a minority population of spheres larger than the great majority of the spheres in the suspension. Then the desolvation agent will be added, followed by a third step of adding a stabilizing concentration of crosslinking agent which will prevent the spheres from resolubilization upon removal or dilution of the desolvation agent. Again the site on the protein molecules available for binding by the crosslinking agent before the formation of the sphere and after the formation of the sphere may be different from all the other methods mentioned in this section.

5. “BiMid-link” means the crossing linking agent is added in two separate steps. The first step, like the Bi-link method, involves the addition of a low sub-stabilizing concentration of crosslinking agent to the protein solution for a short time. The second step involved the addition of a stabilizing concentration of crosslinking agent which had been premixed with the desolvation agent. The advantage of this method is that the spheres formed do not have any detectable minority population of spheres of unusual size compared to the great majority of spheres formed and there is one fewer step of addition or mixing compared to the Bi-link method. Since the crosslinking agent had been pre-mixed with the desolvation agent, the mixture was added together to the protein solution (pre-treated with the sub-stabilizing concentration of crosslinking agent) to form spheres. As discussed above, the site of binding by the crosslinking molecules on the protein molecules may be different from those produced by the other methods mentioned in this section.

### Experiment Five

### Binding of Fibrinogen Molecules to Spheres Produced by Different Methods

Purpose: To evaluate if the spheres prepared by the novel Post-link and the novel Mid-link method can both bind fibrinogen by mixing with a fibrinogen solution, without the need to add additional crosslinking agents and without resulting in the formation of aggregates in the fibrinogen-containing suspension

Rationale: Preliminary experiments had showed that spheres prepared by the Pre-link and Post-linked method required at least 4 minutes before the crosslinking agent could stabilize the spheres with maximal effect. Mid-link method needed more than 8 minutes. Other preliminary experiments had suggested that glutaraldehyde molecules attach to protein molecules very quickly and the reaction would have completed within 5 minutes (data not shown here). To further minimize any effect from glutaraldehyde left in the suspension, fibrinogen molecules were added in this experiment to the turbid suspension at least 10 minutes after appearance of turbidity in the preparation when little or no “still reactive” glutaraldehyde molecules are present.

Commercial supplies of fibrinogen are typically lyophilized power formulations containing a high concentration of salt (e.g. 15% sodium citrate and 25% sodium chloride, see Product F3879 from Sigma-Aldrich Co.) Addition of such a high concentration of salt may lead to aggregation of spheres even though the preparation may be stable in the absence of such added salt derived from the fibrinogen solution. This experiment aims to evaluate the proper dilution of commercial supplies of fibrinogen which would still allow enough fibrinogen to be bound to spheres to have medicinal value without causing aggregation of already formed spheres in the suspension.

Material and Methods: HSA 25% was purchased from Alpha Therapeutics Corp, Glendale and diluted with water to 3% without the addition of detergents or other surfactants.

Human Fibrinogen powder was purchased from Sigma-Aldrich Co. and dissolved in normal saline (0.9% sodium chloride) to 1 mg clottable protein/ml before mixing with sphere preparations at a ratio of 0.5 ml to 1.0 ml of fibrinogen solution per 1.0 ml of sphere suspension. Fibrinogen supplied by other suppliers are expected to be similarly effective.

Post-link albumin spheres were prepared as follows which follow the steps as closely as possible as described for TS1 in U.S. Pat. No. 6,264,988 B1 except (1) no detergent was added to the protein solution, (2) the solutions were mixed in rigid plastic tubes instead of inside a silicone tubing system, (3) a different ratio of the volume of fibrinogen solution added per volume of the sphere suspension was used here as described above.

(a) The novel “Postlink” method: 400 microliter of HSA 3% (diluted with water from the 25% commercial HSA) was placed in a polypropylene microcentrifuge tube, then 640 microliter of ethanol (70% in water) was added and the mixture turned turbid. After 5 minutes, the suspension was added 52 microliter of GL (6.5 mg/ml.) Final concentration of GL in the suspension was 0.31 mg/ml. Ten minutes after appearance of turbidity the fibrinogen solution was added at 0.5 volume per volume of sphere suspension.

(b) The novel “Midlink” method: 400 microliter of HSA 3% (diluted with water from the 25% commercial HSA) was placed in a polypropylene microcentrifuge tube, then 640 microliter of a solution (70% of ethanol in water, also containing 0.5 mg GL/ml) was added. The mixture turned turbid immediately. Final concentration of GL in the suspension was 0.31 mg/ml. Ten minutes after the appearance of turbidity the fibrinogen solution was added at 0.5 volume per volume of sphere suspension.

Both Post-link and Mid-link sphere preparations were centrifuged to remove the ethanol and any residual crosslinking agent or fibrinogen in the supernatant. The pellet was resuspended in normal saline.

To evaluate if fibrinogen coated spheres could form thrombin-induced aggregates in vitro, the method previously described was used. Aggregation of particles under “sub-minimal” concentrations of soluble fibrinogen was previously described in U.S. Pat. No. 6,391,343 B1 “Fibrinogen-Coated Particles for Therapeutic Use” Column 19, line 47-60. Essentially, fibrinogen solutions at low concentrations that normally do not form a visible clot (“sub-minimal” concentration) on addition of thrombin (3 units per ml) were mixed with Control Spheres (CS) or spheres previously coated with fibrinogen. Then thrombin was added. CS do not have fibrinogen on their surface and had been shown in the prior art not to form aggregates on the addition of thrombin to the mixture under these conditions. Spheres previously coated with fibrinogen, however, will form aggregates when suspended in a “sub-minimal” concentration of fibrinogen, after the addition of a thrombin solution.

Result: Addition of a fibrinogen solution (diluted with normal saline) to the sphere suspensions under the conditions of this experiment did not result in aggregate formation from the salt introduced with the fibrinogen solution.

Both fibrinogen-coated-Post-link spheres and fibrinogen-coated-Mid-link spheres form aggregates in the presence of a sub-minimal concentration of fibrinogen, after the addition of a thrombin solution, as described. The data showed that Mid-link spheres could bind fibrinogen to a similar extent as the Post-linked spheres; and both sphere preparations may be effective in treatment of thrombocytopenic animals.

Control Post-link spheres and control Mid-link spheres, both without added fibrinogen to coat the spheres before mixing with the sub-minimal concentration of fibrinogen solution did not form thrombin-induced aggregates under these conditions.

Comments: Although the fibrinogen solution used in this experiment had not been subjected to pressure for the purpose of inactivation of any infection agents in the solution, it is expected that fibrinogen solution that had been treated by pressure as described in Experiments One to Four can be used and will produce spheres coated with fibrinogen in the absence of active infectious agents.

Although the previous experiments used pressure up to 600 MPa, it is conceivable that pressures more than 600 MPa may even be more effective in the inactivation of infectious agents.

Data from preliminary experiments (not shown here) had indicated that binding of GL to protein molecules could complete in less than 5 minutes. Since the fibrinogen molecules were mixed with the spheres in this experiment after 10 minutes of the appearance of turbidity, the binding of fibrinogen molecules to spheres probably did not require or depend on the presence of still reactive, residual amount of crosslinking agents (i.e. any leftover from what was needed to stabilize spheres against resolubilization.) The attachment of fibrinogen molecules to these spheres could be non-covalent.

Fibrinogen-coated-Post-link spheres prepared by the prior art in the presence of a detergent in the protein solution (such as TS 1) could form thrombin-induced sphere aggregates in vitro in sub-minimal concentrations of fibrinogen. Those spheres could also form co-aggregates with human platelets after the addition of aggregation agents such as ADP or collagen in vitro (FIG. 13B, in U.S. Pat. No. 6,264,988 B1.) Therefore, the ability of fibrinogen-coated-spheres made by both the novel Post-link and the novel Mid-link method to form thrombin-induced sphere-sphere aggregates (from single spheres produced by the Mid-link methods) in the presence of sub-minimal concentrations of fibrinogen suggested that fibrinogen-coated-spheres made with these two novel methods would be capable of forming similar co-aggregates with human platelets in vitro and also in vivo.

The failure of control spheres, made by either the novel Post-link method or the novel Mid-link method to form thrombin-induced sphere aggregates suggested under these conditions of low soluble fibrinogen (“sub-minimal”) concentrations, both kinds of spheres either did not bind or did not bind enough fibrinogen molecules to be effective in forming sphere-to-spheres aggregates after the addition of thrombin.

Conclusion: The novel “Mid-link” method could produce spheres approaching the size of natural platelets (which are about 2 micron) without the co-production of large spheres (larger than 5 micron.) The binding of fibrinogen to spheres at a time point when the crosslinking agent would have been exhausted (from having completely bound to the albumin molecules, either in the sphere form or in the residual soluble form) suggested that the binding of fibrinogen to spheres needed not be covalent for the combination to be effective in providing medicinal value. Fibrinogen treated with pressure to inactivate infectious agents are equally effective in coating spheres as fibrinogen molecules not treated with pressure.

### Experiment Six

### Effect of High Pressure on the Efficacy of Sphere Suspensions Prepared by the Novel Post-Link and the Novel Mid-Link Method

Purpose: (1) to see if high pressure would destroy the sphere suspension during pressurization. (2) to evaluate the efficacy of such pressure-treated sphere suspensions.

Material and Method: The sphere preparation (both Post-link method and Mid-link method) manufactured in Experiment 5 which had been coated with fibrinogen were further processed as described below.

An excipient (LMG) was prepared by dissolving 27 gram of lactose, 27 gram of maltose and 12 gram of glycine (all purchased from Sigma) in 300 ml of water. The final volume after all the sugars and amino acids were dissolved exceeded 300 ml. The solution was filtered with a 0.2 micron filter before mixed one part by volume per 3 parts by volume of sphere suspension.

Terminal Sterilization: After the sphere suspensions (containing excipient) were dispensed (10 ml) into the plastic bottles (such as the LifeShield plastic vials sold by Hospira, Inc, Lake Forest for their Sterile Water for Injection, USP, or any container made of poly olefin, a copolymer of ethylene and propylene or any container that does not leak or break under pressure) the gray butyl stoppers were placed tightly and an aluminum flip-off cap (purchased from Kimble) was applied. The bottles were pressurized using standard high pressure equipment such as those described in the literature. Starting temperature in the tank was 39 degree F., highest run temperature was 79 degree F. Extremely high hydrostatic pressure (600 MPa) was applied, average ramp-up time was about 2:12 minutes. Three consecutive runs of 1 minute each were done, with about 5 minutes in between each run to allow time to reset and rechill the tank water. After terminal sterilization, a portion of the plastic vials containing sphere suspensions were kept at room temperature, others at refrigeration temperature, and the rest were frozen at minus 18 degree C.

Evaluation in thrombocytopenic rabbits were as described in prior art.

Results: The overall appearance to the unassisted eye of the sphere suspension inside the plastic bottle after pressure treatment did not change from before pressurization. Inspection under a phase microscope revealed no discernable change in size. There remained no aggregates or clumps in the suspension of spheres. At this high pressure of 600 MPa, it is expected all common bacteria and viruses would be killed.

All the contents inside the treated vials appeared to have no change by visual and microscopic inspection after storage for at least 7 months under the respective conditions.

The data in thrombocytopenic rabbits showed that the fibrinogen-coated spheres were effective in shortening the bleeding time (BT) of severely thrombocytopenic rabbits.

Conclusion: Treatment with pressure up to 600 MPa did not appear to have affected the medical benefit of spheres prepared by the various novel methods followed by coating in vitro of a fibrinogen solution. At this pressure, all commonly known infectious agents would have been inactivated.

### Experiment Seven

### Albumin Spheres Coated with Multiple Human Clotting Factors in Vitro

Purpose: To evaluate if spheres exposed to human plasma can simultaneously bind multiple clotting factors from the plasma.

Rationale: In previous experiments, fibrinogen (also known as Factor I) was purchased from Sigma, e.g. F3879 which contained about 60% protein by weight, of which over 80% of the protein is clottable; the remainder being sodium citrate and sodium chloride. The powder was typically dissolved in normal saline and added to the suspension of spheres to achieve coating of fibrinogen on the surface or imbedding within the matrix of the spheres.

The present experiment aims at evaluating whether more than one clotting factor could bind to spheres spontaneously when spheres were exposed in vitro to human plasma containing the full complement of coagulation factors. Although the plasma used here has not been treated with high pressure for the purpose of inactivating any infectious agents present, it is expected that plasma, or any combination of coagulation factors in solution, that had been treated with high pressure can be effective in binding to albumin spheres.

Material and Methods: The novel “Pre-link” method without the addition of surfactants was used. HSA 25% purchased from supplier A was diluted with water to 10% without addition of detergents or any other surfactants. To 4 ml of this protein solution in a tube, 4 ml of GL (1.6 mg/ml dissolved in water) was added and mixed well by shaking. After 30 seconds, 12 ml of ethanol (70% in water) was added and the mixture turned turbid. The room temperature was about 21 degree C.

Plasma was obtained from a healthy volunteer after removal of all cellular elements from the heparin-anticoagulated whole blood. The plasma contained 2.17 mg of fibrinogen/ml; and normal ranges of vonWillibrand Factor (vWF) and Factor IX.

Fibrinogen concentration was measured by a competitive immuno-assay. Fibrinogen standards purchased from Sigma-aldrich were diluted to a range of 0 to 5 microgram/ml with normal saline containing 10% Blocking Agent (purchased from Pierce; the solution was called NSB in these experiments.)

Spheres to be assayed for their bound-fibrinogen content were likewise diluted to a range of expected fibrinogen concentrations (in the sphere-bound form) suitable for the assay. Goat anti-human-fibrinogen antibody (called GAF here) and rabbit anti-Goat-IgG linked to peroxidase enzyme (called RAG here) were purchased from Sigma-aldrich and diluted to 1:3000 and 1:2000 solutions, respectively (with NSB.) An aliquot of antigen (either standard solution, or spheres; typically 25 microliter) was mixed with 100 microliter of GAF (containing excess antibody with respect to the added antigen.) After incubation, 100 microliter of the mixture was added per well in a 96-well plate. The wells had been precoated with a saturating concentration of fibrinogen. The excess GAF (leftover after some had bound to the fibrinogen on the spheres) would then bind to the fibrinogen pre-bonded on the plastic well. After adequate rinsing, 100 microliter of RAG was added. After further adequate rinsing, the substrate for peroxidase was added to generate a yellow color reaction. The sample with the highest fibrinogen concentration would have removed the largest amount of GAF (from the excess concentration) and thus have the least leftover to bind to the plastic well. Therefore, the higher the fibrinogen content (whether in the soluble form or attached to a sphere surface or interior) in the sample, the lighter the color in the well. Comparison of the color optical density (in a spectrophotometer) with the color optical density of standard solutions generated the concentration of fibrinogen in the sample of interest.

Since there are no commercial supplies of purified human vWF or human Factor IX available to bind to plastic wells (as was possible with fibrinogen) the assay of these clotting factors (attached to spheres) required an indirect method of first “converting” the specific human antigen to a rabbit IgG marker. Rabbit anti-vWF (F 3520) and rabbit anti-Factor IX (F0652) and rabbit non-specific IgG (I5006) were purchased from Sigma-aldrich. Appropriate concentrations of these respective antibody solutions were prepared by dilution with NSB. Then 100 microliter of the respective antibody was mixed with 100 microliter of spheres. Subsequently the excess (still soluble) antibody was removed by centrifugation of the sphere suspension. The spheres were resuspended in normal saline, and they were by now coated with the specific rabbit antibody, if the spheres had the vWF or Factor IX to start with. This treatment converted the amount of specific antigen on the spheres (whether vWF or Factor IX) to an equivalent amount of a generalized antigen of rabbit IgG bound to the spheres.

The resuspended spheres were then subjected to a competitive immunoassay to measure the amount of rabbit IgG bound to the spheres (via the human vWF or Factor IX on the spheres). The antibody used was a goat anti-rabbit antibody (GAR, in excess amounts) which was already linked with a peroxidase enzyme. Left over GAR (not bound up by rabbit IgG on the sphere) was then added to plastic wells pre-bonded with non-specific rabbit IgG. For standard solutions, non-specific rabbit IgG was diluted to a range from 5 to 200 microgram/ml to react with GAR. The amount of GAR bound on the plastic well surface was measured by the addition of a peroxidase substr ate.

The spheres were subjected to treatment in high pressure as described in Experiment 6.

Results: Aliquots of albumin sphere suspensions (200 microliter) prepared with the “Pre-link” method as described were mixed (within 20 minutes after addition of ethanol) with the donor's plasma (diluted with water to achieve a fibrinogen concentration of 1.5 mg/ml in plasma, 160 microliter.) For comparison, another aliquot of the same suspension (200 microliter) was mixed with 160 microliter of purified fibrinogen (also 1.5 mg/ml.) After mixing, the suspension contained about 6 mg of spheres/ml.

The fibrinogen content on spheres coated with plasma and with the purified fibrinogen solution was found to be 20.4 and 18.1 ug fibrinogen/mg spheres, respectively.

The amount of specific rabbit antibody bound for the VWF and the Factor IX was found to be 5.2 ug per mg sphere and 0.47 ug per mg sphere, respectively, in spheres that had been exposed to plasma under the experimental conditions used. Spheres that had not been exposed to plasma had no bound VWF or Factor IX.

Microscopic examination of the sphere suspensions after treatment by pressure showed no difference compared to those before pressure treatment.

Comments and Conclusions: The data showed that endogenous fibrinogen molecules from plasma could bind spontaneously to spheres with the same efficiency as purified fibrinogen preparations obtained from commercial sources. When plasma was used, additional coagulation factors could bind simultaneously. In this experiment only fibrinogen, vWF and Factor IX were studied (as examples of coagulation factors) because antibodies to these factors were commercially available. It is expected that other coagulation factors or even non-coagulation factors, protein or non-protein molecules could bind to the spheres when spheres were mixed with whole blood.

The fact that 0.47 ug equivalent of rabbit IgG (anti-Factor IX) was bound per mg sphere compared to 5.2 ug equivalent of rabbit IgG (anti-vWF) does not mean that fewer IX Factor molecules were bound per mg sphere as compared to vWF molecules. The specificity of binding of the specific antibody (ug of antibody binding to one mg of antigen) to the respective antigen was not known and can be very different for these two antibodies and antigens.

The “Pre-link” method in this experiment was novel because no detergent was added to the protein solution before addition of the desolvation agent. Also a short “crosslinking agent reaction time” of 30 seconds with the protein molecules was used. Compared to the GL interaction time used in disclosed previous patents, 30 seconds was a short time. The suspension contained no spheres of larger than 5 microns and no aggregates.

Although only the novel method of “Pre-linked” spheres were tested in this experiment, it is expected that spheres produced by the novel method of “Post-link” and “Mid-link” and “Bi-link” and “BiMid-link” (all without added surfactant) are all capable of binding multiple coagulation factors and other biological molecules or drugs upon contact with plasma in vitro and in vivo.

It is expected that spheres containing a combination of multiple coagulation factors may have at least comparable or even superior medical efficacies as compared to spheres containing only bound fibrinogen.

Extreme hydrostatic pressure sufficient to inactivate all commonly known infectious agents did not appear to affect the medical efficacy or safety of the sphere suspensions prepared by the various novel methods disclosed in this Invention.

### Experiment Eight

### Synthesis of Albumin Spheres Using the Bi-Link Method Followed with Coating with Various Concentrations of Fibrinogen

Purpose: To manufacture a number of sphere preparations containing increasing concentrations of fibrinogen using the Bi-link Method and a supplier other than Alpha Therapeutics, California, for the purpose of evaluating whether a minimal amount of bound fibrinogen on the spheres is needed for improvement of bleeding time in thrombocytopenic rabbits.

Rationale: Yen disclosed a detail description of manufacturing spheres (U.S. Pat. No. 6,264,988 B1 “Fibrinogen-Coated Microspheres) coated with fibrinogen which resulted in improvement of bleeding time in thrombocytopenic rabbits (FIG. 4, 5, 6.) The human serum albumin (HSA) used to produced spheres in that disclosure was purchased from Alpha Therapeutics, Calif (Supplier A, Column 10, line 1). It was not clear if the beneficial properties were the specific results of using HSA from this supplier as a source material. Preliminary results (to be reported elsewhere) had shown that Baxter Healthcare Corp's product (“Buminate”) appears to differ the most (in terms of chloride and bicarbonate concentrations) from the HSA supplied by Alpha Therapeutics. Therefore, Buminate was used to produce particles in the present experiment, which would be further evaluated (described in Experiment 9) in thrombocytopenic rabbits.

In addition, previous fibrinogen-coated albumin spheres had all been prepared in the presence of a surfactant to prevent aggregate formation. It was not clear if the presence of such a chemical, specifically sodium tetradecyl sulphate (STS) had any effect on the surface properties or other properties of the spheres which might specifically result in or contribute to their efficacy. To decrease any confusion, the Bi-link spheres in this experiment were prepared in the presence of STS, with or without fibrinogen, even though the novel method presented in this Invention does not require the presence of STS.

Also, U.S. Pat. No. 6,264,988 B1 disclosed a method of producing spheres by using various pumps to deliver the respective reagents through a silicone tubing system to critical mixing points for mixing. It was designed for production of massive amounts of sphere suspensions. The present experiment by contrast, achieved mixing of smaller quantities of reagents in rigid plastic tubes or glass flasks.

Materials and Methods: Both HSA 25% and human fibrinogen were purchased from Baxter Healthcare Corp. Glutaraldehyde (GL) was chased from Electron Microscopy Science (EM grade, Port Washington, Pa.) Sodium Tetradecyl sulphate (STS 27%, Niaprof 4, which is the same anionic surfactant formerly produced by Union Carbide under the Tergitol name) was purchased from Sigma, St. Louis.

To approximate as much as possible the concentration of reagents at the mixing junctions as the disclosed prior art of U.S. Pat. No. 6,264,988 B1, the following steps were used for the present experiment:

(a) Preparation of 50 ml of albumin solution containing a detergent with the correct concentration of salt: To a 50 ml polypropylene tube 6.25 ml of water was first added, followed by 5 ml of STS (0.2 mg/ml diluted in water), followed by 3.75 ml of a ten-fold saline solution (90 mg of sodium chloride/ml) and finally 35 ml of Buminate (25%.) This solution contained the appropriate amount of STS and sodium chloride in the protein solution ready for use in the next step and was called snHSA.

Therefore, the concentration of the constituents in snHSA (before addition of GL and other reagents) was as follows: HSA (17.5%); STS (0.02 mg/ml); added sodium chloride (6.75 mg/ml, not counting any cations or anions contributed from the stock 25%-HSA)

(b) The sub-stabilizing concentration and the stabilizing concentration of GL were prepared by dilution with water a stock solution of GL (10%) to 0.1 mg/ml (50 ml prepared), and 12.5 mg/ml (10 ml prepared), respectively.

(c) Ethanol was prepared by dilution with water to 70% in a 500 ml glass flask. Because of the relatively large volume needed, the total volume was added to the protein solution in two equal aliquots, with thorough mixing in between to prevent local areas of high alcohol concentrations within part of the solution mixture.

(d) Solutions of human fibrinogen (each 10 ml) were prepared by dilution of a stock fibrinogen solution (2%) with normal saline to achieve concentrations of 2.0, 1.75, and 1.5 mg/ml, respectively. This concentration refers to the concentration of fibrinogen in the solution before addition to the turbid sphere suspensions (added at a ratio of about 0.2 volume of fibrinogen solution per volume of turbid sphere suspension.)

Each of the above components was mixed thoroughly by shaking after each step at room temperature (19 C to 23 C acceptable.) The time indicated in each step was actual time following time zero, not the time-interval from the previous step. The step-by-step procedure of mixing was as follows:

For Preparation 8-A: (1) 6.2 ml of snHSA was removed from the stock solution and added to a sterile 50 ml polypropylene tube; (2) At time zero, 6.2 ml of the sub-stabilizing concentration of GL was added; (3) at 15 seconds, 10.5 ml of Ethanol (70%) as the desolvating agent was added; a slight turbid appearance could be observed in part of the solution which would quickly redissolve (or clarify) upon shaking of the polypropylene tube (because local high concentrations of alcohol was redistributed by the improved mixing); (4) at 30 seconds, another 10.5 ml of Ethanol (70%) was added; the suspension became completely and stably turbid; (5) at 2 minutes, 1.3 ml of the stabilizing concentration of GL was added; (6) at 5 minutes, 4.1 ml of 10-fold saline (90 mg sodium chloride/ml) was added to bring the suspension close to physiological isotonicity; (7) at 6.5 minutes, 8.3 ml of fibrinogen solution (2.0 mg/ml) was added.

For Preparation 8-B and Preparation 8C: the above procedure was repeated, except the fibrinogen concentration was 1.75 and 1.50 mg/ml, respectively.

For Preparation 8-D which was the control sphere suspension (CS), step (7) was omitted.

After the 4 different sphere preparations were manufactured, they were dialyzed 3 times against at least 10 fold excess of distilled water to remove the desolvation agent, any dialyzable molecules and the detergent. An appropriate excipient (the LMG solution described in a previous experiment) comprised of maltose, lactose and glycine was added to facilitate storage by freezing at −18 degree C.

Results: The concentration of spheres (in the samples after thawing the frozen preparations) in Preparation 8-A, 8-B, 8-C and 8-D were 4.6, 4.6, 3.0 and 7.1 mg of spheres per ml suspension, respectively; the amount of fibrinogen attached were 3.5, 2.7, 3.1 and zero ug of fibrinogen per mg sphere, respectively.

The average size of the spheres in all 4 preparations were similar, being about 0.8 micron in diameter and did not have any spheres or particles larger than 5 micron. The preparations all appeared homogeneous in size distribution.

Comments and Conclusions: The data showed that albumin spheres made in the presence of STS, with HSA from a supplier other than Alpha Therapeutics (in this case Baxter Healthcare Corp) using the novel Bi-Link method was capable of binding fibrinogen. Since the amount of fibrinogen bound was 3.1 ug/mg spheres when the fibrinogen solution used was 1.5 mg/ml, which was comparable to the 2.7 ug of fibrinogen bound per mg spheres when the fibrinogen solution used was 1.75 mg/ml, the data suggested that under these conditions of mixing, 1.5 mg of fibrinogen/ml might have reached a saturating concentration for use in coating the spheres in the present manufacturing procedure.

The data showed that spheres could be produced by the Bi-link method without the addition of a detergent in the protein solution and the suspension did not have any detectable amount of large spheres or particles (greater than 5 micron) even though the initial concentrations of reagents were similar to those used in the prior disclosed art which resulted in a sub-population of large spheres.

The data also showed that the desolvation agent could be added in two divided steps and still resulted in biologically useful and safe sphere suspensions.

Although the spheres in this experiment had not been subjected to high pressure for the purpose of inactivation of any infectious agents, it is expected that treatment with pressure will not affect the medicinal benefit of such spheres.

### Experiment Nine

### Evaluation of the Medical Efficacy of Albumin spheres Coated with Fibrinogen Before and After Pressure Treatment

Purpose: To evaluate the medical benefit of intravenous infusion of albumin spheres prepared by the Bi-Link method, comparing spheres coated with fibrinogen before and after high hydrostatic pressure treatment

Rationale: Previous in vivo studies using thrombocytopenic rabbits to demonstrate efficacy in the improvement in bleeding time (BT) or bleeding volume (BV) involved spheres made with the Post-link method in the presence of a surfactant (STS). This experiment was design to evaluate if spheres prepared by the Bi-link method had similar efficacy. In addition, the effect of high pressure on these spheres were studied.

To make spheres prepared by the Bi-link method as similar as possible to those made with the older disclosed Post-link method, the spheres in this experiment were prepared in the presence of STS, even though in this Invention the novel Bi-link method does not require the presence of any added surfactants or detergents. This was done in view of the fact that the effect of an added detergent or surfactant on the medical efficacy of the sphere was unknown. In case the Bi-link method produced spheres which would not shorten the Bleeding Time of thrombocytopenic rabbits, there would be one less confusing factor (that of the potential effect of a surfactant).

The data in this experiment showed that Bi-linked spheres prepared in the presence of a surfactant were effective in vivo. Subsequent experiments to be described below will show that the presence of a detergent in the protein solution was not a factor in the efficacy of the spheres prepared by this novel Bi-link method. Spheres prepared without the added surfactant or detergent in the protein solution were effective in vivo.

Material and Methods: The method of production of Bi-Link spheres was described in Experiment 8. Specifically, suspensions containing spheres with 3.6 ug fibrinogen/mg sphere before pressure treatment (Preparation 8-A, with 4.6 mg spheres per ml suspension) and after pressure treatment (Preparation 8-X with 4.6 mg spheres per ml suspension) were infused into thrombocytopenic rabbits.

Method of using BT to evaluate various platelet substitute products was described in “Novel Platelet Products and Substitutes” by D. H. Lee and M. A. Blajchman (Transfusion Medicine Reviews, vol 12, No 3, July 1998, pp 175-187.) Rabbit platelet count was done by a manual method and was not affected by the protein spheres infused into the animals.

Results: Table 9 showed the platelet counts (×10 billion/L) at various times and the BT (in seconds.) Rabbits that continued to bleed over 900 seconds had the wound compressed temporarily to stop the bleeding. Therefore BT over 900 seconds would be interpreted to indicate a lack of efficacy of the infused product.

All 3 rabbits received 6 ml of sphere suspension/kg weight, intravenously, to ensure equal volumes of fluid were infused. Rabbits 1, 2, 3 were all infused spheres with fibrinogen without pressure treatment (Preparation 8-A). The results on rabbits infused with pressure- treated spheres (Preparation 8-X) were similar (data not shown).

TABLE 9
Platelet Counts of irradiated thrombocytopenic rabbits and their Bleeding
Time at 1 hr and 24 hr post-infusion of spheres

| | | Platelet | Platelet | Platelet | Platelet | Bleeding | Bleeding |
|---|---|---|---|---|---|---|---|
| Rabbit | Weight, | Ct, Pre- | Ct, 0.5 hr | Ct, 1 hr | Ct, 24 hr | Time, 1 hr | Time, 24 hr |
| # | kg | infusion | post-infusion | post-infusion | post-infusion | post-infusion | post-infusion |
| 1 | 3 | 38 | 19 | 20 | 11 | 810 | 540 |
| 2 | 2.8 | 22 | 24 | 17 | 22 | >900 | 489 |
| 3 | 3.1 | 28 | 31 | 36 | 34 | 735 | >900 |

The data showed that the weight and platelet counts were comparable for the 3 rabbits, with Rabbit 1 being the most thrombocytopenic at the 24 hr time point.

Rabbit 1 showed that Preparation 8-A clearly had efficacy lasting up to (and probably beyond) 24 hours after infusion of the fibrinogen-coated spheres. Rabbits 2 showed that what appeared to be an ineffective dose at 1 hour post-infusion was clearly effective by 24 hours. The reason for the delay was not clear. Efficacy might have been demonstrated soon after 1 hour post-infusion but BT at such a time point (e.g. at the 4-hour time point) was not performed. Rabbit 3 showed that what was effective at 1 hour post-infusion at this dose was not observed at 24 hour post-infusion. Whether this observation was due to a relatively low dose (compared to the effective dose which needs to be determined) which could be improved was not clear from this experiment. But overall, the combined data from rabbit 1 to 3 showed that, as expected, spheres coated with fibrinogen in vitro as a part of the synthesis procedure, even when prepared by this new Bi-link method (but in the presence of STS) was effective.

Comments: Data from rabbits infused with spheres not treated with high pressure to inactivate any infectious agents but coated in vitro during synthesis with fibrinogen showed that they were effective in the treatment of thrombocytopenic rabbits. Data from rabbits infused with similar sphere suspensions after high pressure treatment show similar efficacy. High pressure did not appear to harm or damage the sphere suspensions.

### Experiment Ten

### Production of Ultra-Small Spheres by a Novel BiMid-Link Method and Efficacy in Animals

Purpose: To evaluate if spheres could be produced by a novel two step crosslinking method without the addition of surfactants or detergents in the protein solution, the first step comprising the mixing of a sub-stabilizing concentration of crosslinking agent with the protein solution, the second step involving the addition of a mixture containing a crosslinking agent at a stabilizing concentration which had been pre-mixed with the desolvating agent. (2) to evaluate the property of spheres formed by this BiMid-link method.

Rationale: Previous experiments had shown that by first mixing the protein solution for about 15 seconds with a sub-stabilizing concentration of crosslinking agent before the addition of the desolvation agent, the protein spheres obtained after the addition of the desolvation agent would be more uniform in size compared to the spheres formed by addition of the desolvation agent directly to the protein solution without using the sub-stabilizing concentration of crosslinking agent. Since the sub-stabilizing concentration of crosslinking agent was not able to hold spheres in the intact form upon dilution of the concentration of desolvation agent, a stabilizing concentration of crosslinking agent must be added after the formation of spheres to stabilize them against re-dissolving.

The present experiment is carried out to test if useful and highly uniform spheres could be formed by: (1) mixing a protein solution with a sub-stabilizing concentration of crosslinking agent for about 15 seconds, (2) then adding the desolvation agent which had been pre-mixed with a crosslinking agent such that upon mixing of this mixture of desolvation agent and crosslinking agent with the pre-treated protein solution, spheres could be formed that are both uniform and stable against re-dissolving, should the desolvation agent be removed or diluted later.

The advantage of using this BiMid-link method is that one fewer step which requires precise timing is needed compared to the Bi-link method. In the Bi-link method, at time zero the sub-stabilizing concentration of crosslinking agent is added; then at time 15 second (plus or minus 5 second) the desolvation agent is added; and then finally at another time point, the stabilizing concentration of crosslinking agent is added. This will require three mixing steps at three definite time-points. In a tubing set, such as described in the prior art (U.S. Pat. No. 6,013,285) three mixing points are needed and at least two respective post-mixing segments of exact lengths must be included to allow the respective time delays before the next ingredient is to be mixed in at the next mixing junction. With this new BiMid-link method, only two mixing junction points are needed.

This method is novel because the protein solution does not require addition of a detergent or surfactant to produce a sphere suspension that is monodisperse and without aggregates and without spheres larger than 5 micron.

The effect of binding a sub-stabilizing concentration of crosslinking agent on protein molecules still in solution is unknown in terms of the physiological properties of the spheres to be subsequently formed. The effect of adding the second dose of crosslinking agent together with the desolvation agent to irreversibly bind the partially-treated protein molecules from soluble form into a solid sphere is also unknown. Therefore this method is novel and the sphere suspension formed will need to be tested in thrombocytopenic rabbits.

This experiment will include spheres produced by an addition step of coating with fibrinogen in vitro as part of the manufacturing steps. Both the spheres coated with fibrinogen after pressure treatment and those without pressure treatment will be evaluated in thrombocytopenic rabbits.

Material and Methods: Aliquots of a 25% solution of human serum albumin purchased from Baxter (Buminate) was diluted with water to achieve a 10% solution, without the addition of surfactant or a detergent. The sub-stabilizing concentration of crosslinking agent was prepared by dilution of the 25% glutaraldehyde (GL) solution purchased from Sigma (G6257) with water to an initial concentration of 0.15 mg per ml. The desolvation agent was ethanol diluted with water to 60% (vol per vol) premixed with GL at 0.5 mg/ml. Fibrinogen was dissolved to result in a solution with 0.4 mg fibrinogen per ml in a solution containing sodium tetradecyl sulphate (STS) at 1 mg per ml in water. The STS was used to facilitate the solubility of the fibrinogen molecules and not in any way designed to affect the formation of spheres or their stability after synthesis. The excipient of dextrose solution was prepared by dissolving 22 gram of dextrose (purchased from Sigma, D9434) in 100 ml of water.

A portion consisting of 2.1 ml of the 10% albumin solution was added to a 50-ml polypropylene tube at room temperature ranging from 20 to 24 deg C. At time zero, 1.05 ml of the sub-stabilizing concentration of GL was added to the tube and the mixture well mixed by shaking. At time point of 15 seconds, 31.5 ml of the desolvation agent (EG60 containing 0.5 mg GL per ml) was added. The contents were again quickly and well mixed. At time point 30 seconds (counting from time zero) 3.15 ml of the fibrinogen solution was added. Again the mixture was well shaken immediately. After 1 to 2 hours, 12.6 ml of a dextrose solution was added as excipient. This preparation was called Preparation 10-F.

For control, 3.15 ml of water was added in stead of the 3.15 ml of fibrinogen solution at the same time-point of 30 seconds after the addition of the sub-stabilizing concentration of GL (time zero). This control preparation was called Preparation 10-B.

The method of pressurization was similar to that described in previous experiments.

Results: Microscopic examination of both. Preparation 10-F and 10-B showed that the spheres were about 0.1 micron in diameter and very uniform; without any aggregates and without any spheres larger than 1 micron.

Evaluation of these sphere suspensions in thrombocytopenic rabbits showed that Preparation 15-F, without pressure treatment and after pressure treatment were both effective in reducing the Bleeding Time in these animals. Table 10 showed only the result of the preparation without pressurization. The results of this Preparation after terminal sterilization was similar.

TABLE 10
Platelet count and Bleeding Time at various time points in
thrombocytopenic rabbits.

| | | | | | Plt Ct, | Plt Ct, | Plt Ct, | BT, | BT, | |
|---|---|---|---|---|---|---|---|---|---|---|
| Rabbit | Weight | | Dose, | Plt Ct, | 0.5 h | 2.5 h | 24 h | 2 h | 24 h | |
| No | Kg | Prep | mg/kg | Pre | Post | Post | Post | Post | Post | HCT % |
| 47 | 2.9 | 10-F | 4 | 33 | 41 | 19 | 40 | 735 | 240 | 24 |
| 48 | 2.6 | 10-F | 4 | 33 | 32 | 33 | 30 | 715 | 124 | 27 |
| 49 | 2.7 | 10-F | 4 | 15 | 20 | 39 | 44 | 375 | 225 | 25 |
| 50 | 2.9 | 10-F | 1.3 | 42 | 50 | 24 | 41 | >900 | >900 | 24 |
| 51 | 2.9 | 10-F | 1.3 | 23 | 27 | 46 | 42 | 745 | 200 | 25 |
| 52 | 2.8 | 10-F | 1 3 | 50 | 21 | 12 | 28 | 290 | 345 | 28 |

Comments: The data indicated that 4 mg spheres/kg was more effective than 1.3 mg sphere/ml for Preparation 10-F.

Both preparations (one without pressurization and one after pressurization treatment) were injected into rabbits without first removing the alcohol from the preparation and seemed to cause no ill effect on the anesthesized rabbits during the performance of Bleeding Time (BT). However, this Invention envisions the possibility of first removing the ethanol from the sphere preparation before injection into awake animals (such as in clinical practice) to diminish any mental confusion arising from the effect of alcohol. There are many methods available for the removal of alcohol, such as by reverse osmosis, or by diafiltration using various methods including hollow fiber filters such as those used in the wine industry to prepare low alcohol or no-alcohol drinks.

Conclusion: The new BiMid-link method produced spheres which were very uniform and effective in the treatment of bleeding in thrombocytopenic rabbits whether they had been treated with high pressure or not. A dose of 4 mg sphere/ml appeared to be effective.

Defined in detail, the present invention is a method of inactivation of infectious agents in a fluid containing plasma protein and potentially containing at least one infectious agent, comprising: (a) placing said fluid in a container which is resistant to leakage under high pressure; (b) pressurizing said fluid inside said container to a pressure sufficient to inactivate said potential infectious agent; (c) pressurizing said fluid under said high pressure for a time duration sufficient to inactivate said potential infectious agent; and (d) pressuring said fluid under said high pressure at an initial temperature that does not inactivate coagulation factors under said conditions.

Defined broadly, the present invention is a fluid containing plasma proteins which is pressurized to inactivate infectious agents, the plasma proteins comprised of serum albumin.

Defined more broadly, the present invention is a fluid containing plasma proteins which is pressurized to inactivate infectious agents, the plasma proteins comprised of serum albumin and at least one coagulation factor.

Also defined more broadly, the present invention is a fluid containing plasma proteins which is pressurized to inactivate infectious agents, the plasma proteins comprised of at least one coagulation factor.

Defined even more broadly, the present invention is a method of treating a patient lacking sufficient concentrations of at least one coagulation factor by injection or infusion of a fluid containing at least one coagulation factor after the fluid has been pressure treated to inactivate any infectious agent.

Defined even more broadly, the present invention is a method of treating a patient with tendencies to bleed with protein spheres which have been pressurized to inactivate any infectious agent.

Defined even more broadly, the present invention is a method of inactivation of infectious agents in a fluid containing protein spheres and potentially containing at least one infectious agent, comprising: (a) placing said fluid in a container which is resistant to leakage under high pressure; (b) pressurizing said fluid inside said container to a pressure sufficient to inactivate said potential infectious agent; and (c) pressurizing said fluid under said high pressure for a time duration sufficient to inactivate said potential infectious agent.

Of course the present invention is not intended to be restricted to any particular form or arrangement, or any specific embodiment, or any specific use, disclosed herein, since the same may be modified in various particulars or relations without departing from the spirit or scope of the claimed invention hereinabove shown and described of which the apparatus or method shown is intended only for illustration and disclosure of an operative embodiment and not to show all of the various forms or modifications in which this invention might be embodied or operated.

What is claimed is:

1. A protein product comprising monodispersed, ultra-small, non-aggregated, cross-linked protein spheres, with less than 1 percent of the spheres having a diameter greater than one micron, said protein spheres have not been exposed to surfactants and have not been subjected to steps of size-fractionation, said protein spheres effective in pharmaceutical applications.

2. A protein product according to claim 1 where said cross-linked protein spheres are partially cross-linked with a chemical agent at a sub-stabilizing concentration of the agent, followed by a completion of cross-linkage by the same agent on the protein spheres.

3. A protein product according to claim 1 where the median size of protein spheres is within the range of 0.1 to 0.4 microns.

4. A protein product according to claim 1 where the protein is comprised of protein extracted from plasma.

5. A protein product according to claim 1 where the chemical agent is a bivalent chemical cross-linking agent.

6. A protein product according to claim 1 where the protein product has an added excipient.

7. A protein product according to claim 1 where said protein product is effective in the treatment of bleeding problems at a dose of 4 mg proteinsphere per kilogram weight of the patient.
